(19) United States
(12) Patent Application Publication
(10) Pub. No.: US 2009/0264688 A1
(43) Pub. Date: Oct. 22, 2009

(54) CATALYTIC OXY-FUNCTIONALIZATION OF METAL-CARBON BONDS

(76) Inventors: Roy A. Periana, Jupiter, FL (US); William A. Goddard, III, Pasadena, CA (US); William Tenn, Houston, TX (US)

Correspondence Address: PILLSBURY WINTHROP SHAW PITTMAN LLP, ATTENTION: DOCKETING DEPARTMENT, P.O BOX 10500, McLean, VA, 22102, US

(21) Appl. No.: 12/354,318

(22) Filed: Jan. 15, 2009

Related U.S. Application Data
(60) Provisional application No. 61/021,601, filed on Jan. 16, 2008.

(51) Int. Cl. C07C 27/22

(52) U.S. Cl. 568/911

(57) ABSTRACT

The development of compatible functionalization reactions with methyl rhenium(I) species, for integration with the CH activation reaction of hydrocarbons by transition metal alkoxo complexes is described. The invention is applicable to the design of rapid, stable CH activation systems integrated with an oxy-functionalization reaction for selective, low temperature hydrocarbon oxidation catalysts.

### CROSS-REFERENCE TO RELATED APPLICATIONS

The present Application claims priority from U.S. Provisional Patent Application No. 61/021,601 filed Jan. 16, 2008, entitled “Oxidative Functionalization of Low Valent Metal Alkyl Intermediates,” which is incorporated by reference herein in its entirety.

### BACKGROUND OF THE INVENTION

The direct conversion of natural gas or methane to liquid fuels such methanol or methyl esters promises to expand raw feedstock sources for the petroleum and energy industries. However, the current technology capable of converting methane to methyl esters in high conversion (generating >1 M concentrations) and activity is based on electrophilic Pt(II) and Hg(II) CH activation catalysts that require strong acid solvents such as sulfuric acid (H₂SO₄) in order to work efficiently. Such electrophilic catalysts are active in concentrated sulfuric acid because the energy barrier for coordination of methane is low enough to allow CH activation to proceed below 250° C. However, as the reaction proceeds, the acidity of the solvent decreases due to water or methanol ester product formation, thereby decreasing catalyst activity, and CH activation effectively stops below about 85% H₂SO₄. In order to develop the next generation of hydrocarbon oxidation catalysts, new thermally, acidic, and oxidant stable catalysts will be required where the CH activation reaction is not inhibited by water or products.

A move to more water tolerant catalysts systems creates a second problem. Metal-carbon intermediates generated from CH activation reactions with electrophilic metal cations in strongly acidic media have polarized metal carbon bonds that may be depicted as M^{δ-}-^{δ+}CH₃. Such polarization increases the positive charge on carbon, rendering them susceptible to reductive oxy-functionalization by attack of external oxygen nucleophiles on the methyl group. In contrast, with the move to less electrophilic metal complexes, metal-carbon intermediates generated from CH activation reactions in weakly acidic to basic media may be expected to have metal carbon bonds oppositely polarized as M^{δ+}-^{δ-}CH₃. Such polarization increases the negative charge on carbon, rendering them less susceptible to reductive oxy-functionalization by attack of external oxygen nucleophiles on the methyl group. Thus, the pathway for M-R oxy-functionalization that operates for the electrophilic metal cations such as Pt(II), Pd(II), and Hg(II) systems is not available with less electrophilic systems.

### SUMMARY OF THE INVENTION

The present invention describes the use of oxy-functionalization of metal alkyl complexes based on a transalkylation/reductive functionalization (TRF) sequence as disclosed herein for the design and development of new catalysts for the selective, conversion of methane to functionalized products at temperatures below 250° C. When used together with (1) a catalyst that operates by CH activation to generate metal-carbon intermediates, (2) reaction conditions that are compatible with the various reactions and (3), an oxidant that allows the thermodynamically favorable conversion of methane to methanol or methyl esters. Catalyst that operate by the TRF sequence described herein can lead to the design and development of complete system for converting hydrocarbons to derivatized products in basic, neutral, or weakly acidic media.

According to one embodiment, the invention is a chemical process for producing alcohols from metal alkyl complexes. Metal alkyl complexes are intermediates formed from the CH activation of alkanes by transition metal catalysts. In one embodiment, the process consists of first contacting a metal alkyl complex with an alkyl acceptor. Transalkylation then occurs, transferring an alkyl group to an alkyl acceptor. An oxidant, for example an O-atom transfer agent or electron acceptor and O-atom source then reacts with the alkylated acceptor, producing an alcohol and regenerating the alkyl acceptor.

In one embodiment, metal alkyl complexes of the invention comprises a group 8 transition metal, which include but are not limited to rhenium, ruthenium, osmium, rhodium, and iridium. One embodiment of metal complexes include low-valent rhenium carbonyl complexes, optionally substituted with phosphine or amine ligands.

Suitable alkyl acceptors include atoms or molecules which include the elements selenium, copper, iron, nickel, manganese, vanadium, mercury, platinum, palladium, and silver. Suitable selenium-based alkyl acceptors include but are not limited to selenium oxo species such as selenic acid, selenous acid, or selenium dioxide.

The amount of alkyl acceptor present can be stoichiometric or catalytic relative to the methane and oxidant consumed in the reaction. Thus in one embodiment of the process of the invention, the molar amount of the alkyl acceptor is substantially less than the molar amount of the oxidant. In this case, the amount of alkyl acceptor is “catalytic” as it is used and recycled in-situ many times over during the process of alcohol production. An alkyl acceptor receives an alkyl group, is oxidized by an O-atom donor, releases an alcohol derived from the alkyl group, and is regenerated to react with another equivalent of metal alkyl complex.

According to one embodiment, an O-atom donor as oxidant for the conversion of methane can be iodate, periodate, or mixtures of iodine and oxygen. It is thermodynamically favorable to generate iodate (IO₃⁻) from iodide (I⁻) or iodine (I₂) with dioxygen, O₂. Iodine and iodide (I⁻), and iodate species are redox related and interconvertable are plausible under varying pH conditions of the present invention. This would allow the use of catalytic or stoichiometric amounts of these O-atom donors which when recycled by air would allow the overall, economical conversion of methane to functionalized products with air. Oxy-functionalization is a sub-class of a broader class of hetero atom functionalization reactions. Thus in another subclass, N-atom functionalization refers to the derivatization of metal alkyl complexes to yield alkyl amines.

An oxy-functionalization reaction can be coupled to a CH activation catalyst to catalyze the overall conversion of an alkane to an alcohol or alkyl esters under CH activating conditions. Accordingly, another embodiment of the invention is a process for the selective oxidation of alkanes to alcohols which consists of contacting an alkane with a CH activating metal complex and an alkyl acceptor, thereby producing a metal complex comprising an activated alkyl. In the presence of a suitable alkyl acceptor, transalkylation will occur from the CH activating complex to the alkyl acceptor, thereby alkylating the alkyl acceptor. If a suitable oxidant is also present, for example an O-atom donor, the alkyl acceptor releases an alcohol and is regenerated as an alkyl acceptor suitable for reaction with another equivalent of metal alkyl complex and also regenerating the CH activating metal complex.

Suitable CH activating metal complexes include a group 8 transition metal, especially those in oxidation states less electrophilic than Pt(II) and Hg(II), although those metals are not excluded. Suitable CH activating metals include low to medium oxidation states of rhenium, ruthenium, osmium, rhodium, and iridium, although no oxidation state is specifically excluded.

### BRIEF DESCRIPTION OF THE DRAWINGS

FIG. 1 shows a generalized catalytic cycle for the conversion of alkanes (RH=CH₄) to methanol (CH₃OH) via CH activation and transalkylation/reductive functionalization (TRF).

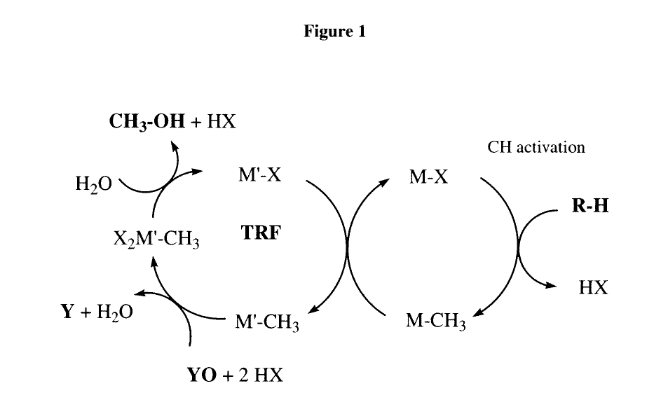

FIG. 2 shows a generalized catalytic cycle for the conversion of alkanes (R-H) to alcohols (ROH) via CH activation and transalkylation/reductive functionalization.

FIG. 3 shows another generalized catalytic cycle for the conversion of alkanes (R-H) to alcohols (ROH) via CH activation and transalkylation/reductive functionalization, using a different transalkylation agent than in FIG. 2.

FIG. 4 shows a transalkylation/reductive functionalization reaction using SeO₃H₂.

FIG. 5 shows a calculated (B3LYP) low energy transitiona state for methyl transfer.

FIG. 6 shows a complete catalytic cycle for the oxidation of methane to produce methanol according to the invention.

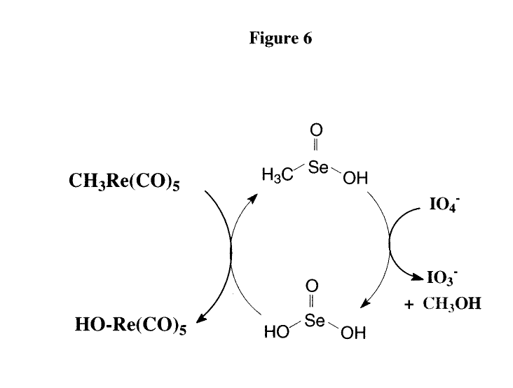

### DETAILED DESCRIPTION OF THE INVENTION

In order to provide a clearer and more consistent understanding of the specification and the claims, the following definitions are provided:

The term “activating” refers in general to causing a chemical species to become reactive towards another chemical species. In a non-limiting example, a catalyst which may be normally inactive or slow to react with the desired species, for example an alkane substrate, may be activated by the addition or via contact with a non-alkane molecule.

The term “activating a C—H bond” refers to a process whereby a C—H bond and a metal ligand complex, MX, react to generate a metal-alkyl complex comprising a metal-carbon covalent bond (M-C). The reaction comprises two steps that contribute to the energy barrier for the overall reaction. The two steps generally involved are, but not limited to: (1) C—H bond coordination to a metal catalyst and (2), subsequent C—H bond cleavage to yield a metal alkyl complex designated M-R. C—H activation as defined herein proceeds without the involvement of free radicals, carbocations or carbanions to generate M-R intermediates. C—H activation which does not include a complete and irreversible conversion to functionalized alkane products can in some case be physically detected by the reversible incorporation of hydrogen isotopes (deuterium or tritium) into an alkane reactant. Thus C—H activation may refer to the ability of a catalyst to catalyze H/D exchange between an alkane or arene reactant and a deuterium source such as for example a protic solvent.

The term “alkane” refers to a non-aromatic saturated hydrocarbons with the general formula CₙH(2ₙ+2), where n is 1 or greater. Alkanes maybe straight chained or branched. Examples include methane, ethane, propane, butane, cyclohexane, cyclooctane. Alkanes may solid, liquid or gas.

The term “arene” refers to an unsaturated hydrocarbon, the molecular structure of which incorporates one or more essentially planar sets of carbon atoms that are connected by delocalized electrons comprising a conjugated π-system. A prototype aromatic compound is benzene. Other examples of arene hydrocarbons are the polycyclic aromatic hydrocarbons comprising more than one aromatic ring.

The term “catalyst” refers to a chemical agent that facilitates chemical processes. In one sense, the term is used to describe a reagent used to activate a hydrocarbon C—H bond. In another embodiment, the term refers to a substance that initiates or accelerates a chemical reaction without the overall catalyst concentration, structure and composition being affected in the overall reaction. According to several embodiments of the invention, catalysts facilitate chemical reactions between hydrocarbons, oxidants, solvents and other components of a chemical transformation. Catalysts themselves are not consumed, rather they continuously react and are regenerated. Coordination catalysts are a class of catalysts that facilitate chemical reactions by bringing together or “coordinating” reactants within the coordination sphere of the central coordinating atom of the catalyst.

The term “coordination sphere” refers to the first sphere of attracting force around the central coordination atom of the catalyst.

The term “catalytic composition” refers to a catalyst and supporting agents such reactants, solvent, oxidant and co-oxidant.

The term “conjugated π-system” refers to a planar organic compound containing two or more conjugated multiple bonds. Arenes as defined herein have conjugated π-systems. Conjugated π-systems may also comprise hetero atoms and metal atoms.

The term “functionalized hydrocarbon” refers to a hydrocarbon wherein at least one C—H bond has been transformed into a carbon functional group bond, a carbon heteroatom bond, where the heteroatom is anything other than H. By way of example only, functionalized methane is methanol. Functionalized benzene is phenol.

The term “Group 7 of the periodic table” refers to the elements manganese, technetium, and rhenium.

The term “Group 8 of the periodic table” refers to the elements iron, ruthenium, and osmium.

The term “Group 9 of the periodic table” refers to the elements cobalt, rhodium, and iridium.

The term “hydrocarbon C—H bond” refers to a covalent bond between hydrogen and carbon localized within a hydrocarbon molecule. A C—H bond may be described in terms of frontier molecular orbital theory as having a highest occupied molecular orbital (HOMO) and a lowest unoccupied molecular orbital (LUMO).

The phrase “hydrocarbon activation is accelerated by solvent” refers to a rate increase due to changes in the concentration or composition of the solvent which is predicted or observed for a C—H bond activation event.

The term “ligand” refers to the set of atoms, ions, or molecules in contact with a metal ion. Ligands comprise the set of atoms, ions, or molecules surrounding a metal ion in the first coordination sphere of the metal. Free ligands may be indistinguishable from solvent molecules.

The term “ligating atom” refers to an atom or atoms comprising a ligand which bind to a metal. The term “ligating atom” is equivalent to “donor atom” in certain embodiments.

The term “linked nitrogen heterocycle” refers to bipyridine, bipyrazine, bipyrimidine and the like.

The term “metal-alkyl covalent bond” refers to an alkyl group bonded to a transition metal or metal complex.

The term “metal alkyl complex” refers to an alkyl group bonded to a metal complex.

The term “N-donor atom” refers to ligand or solvent molecules which binds directly to metals according to certain embodiments of the invention. N-donor atoms may be part of N-donor ligands. Suitable N-donor ligands include nitrogen heterocycles as defined below.

The term “nitrogen heterocycle” refers to organic compounds that contain a ring structure containing nitrogen atoms as part of the ring. They may be either simple aromatic rings or non-aromatic rings. Non limiting examples include pyridine, pyrimidine, and pyrazine.

The term “non-radical producing” refers to a method or process characterized by the absence of free radicals. Such radicals may be oxygen-based, carbon based, or metal based.

The term “O-atom donor” refers to any O-atom donor that has a potential to thermodynamically favorably oxidize methane to methanol at a temperature of 300° C. or lower. Thermodynamic potentials for methane oxidation may be calculated from the equation:

CH₄+YO=CH₃OH+Y

The change in Gibbs free energy for this reaction, ΔGᵣₓₙ, determines whether an O-atom transfer donor has the potential to thermodynamically oxidize methane with favorable thermodynamics. Values of ΔGᵣₓₙ <0 based on calculated or tabulated data for the equation: CH₄+YO=CH₃OH+Y indicate the conversion of methane is feasible. An approximation of the ΔGᵣₓₙ may be obtained by considering the related bond strengths of the reactants and products. On this basis any oxidant (YO) with Y-O homolytic bond strength of less than −90 kcal/mol is a candidate O-atom donor.

The term “O-atom insertion agent”, abbreviated as (YO) refers to an agent or reagent that provides a source of oxygen atoms. A YO reagent reacts selectively, by non-radical pathways. Suitable oxidants give up their oxygen atom and can be reoxidized by O₂ from the air with favorable thermodynamics. These O-atom transfer reagents can insert oxygen directly into a metal-carbon bond to make intermediate methoxide species or they may preferentially react with an intermediate species from a transalkylation. It is also possible that YO reagents can act as oxidants or electron acceptors with the oxygen required for methanol formation provided by another reactant such as water or an oxygenated solvent.

The term “oxidant” refers to a compound that oxidizes (removes electrons from) another substance in a chemical oxidation, reaction, process or method. In doing so, the oxidizing agent, sometimes called an oxidizer or oxidant, becomes reduced (gains electrons) in the process. An oxidizing chemical reaction is a broadly defined and may have several meanings. In one definition, an oxidizing agent receives (accepts) electrons from another substance (reductant). In this context, the oxidizing agent is called an electron acceptor. Broadly speaking, such chemical events occur in two distinct ways which can be described as inner sphere or outer sphere. In another meaning, an oxidant transfers O atoms to the reductant. In this context, the oxidizing agent can be called an oxygenation reagent or oxygen-atom transfer agent. Examples include amine-N-oxide, cupric oxide, iron oxide, periodate (IO₄⁻), iodate (IO₃⁻⁻), vanadate (VO₄³⁻), molybdate (MoO₄²), nitrous oxide (N₂O), hydrogen peroxide (H₂O₂), selenate (SeO₄²⁻), tellurate (TeO₄²⁻), hypochlorite (ClO⁻), chlorite (ClO₂⁻), nitrate (NO₃⁻), and sulfoxide.

The term “oxidation stable solvent” refers to a solvent that is not itself oxidized during any step of a chemical reaction, method, or process.

The term “oxygen insertion agent” refers to an agent which functions as both an oxidant and as a source for an oxygen atom which inserts into a metal-alkyl covalent bond. Examples include Examples include amine-N-oxide, cupric oxide, iron oxide, periodate (IO₄⁻), vanadate (VO₄³⁻), molybdate (MoO₄²⁻), nitrous oxide (N₂O), hydrogen peroxide (H₂O₂), selenate (SeO₄²⁻), tellurate (TeO₄²⁻), hypochlorite (ClO⁻), chlorite (ClO₂⁻), nitrate (NO₃⁻), and sulfoxide.

The term “oxygenated hydrocarbon” refers to a hydroxylated hydrocarbon. Methanol is an oxygenated hydrocarbon (methane).

The term “oxidation resistant ligands” refers a ligand(s) that is not itself oxidized during any step of a chemical reaction, method, or process.

The term “reduced oxidant” refers to an oxidant which has transferred an O atom during or as a consequence of an alkane functionalization process. By way of example, for the oxidant SeO₄²⁻, the reduced oxidant is SeO₃²⁻.

The term “regenerating the catalyst” refers to a step during a process for selective oxidation of hydrocarbons. During this step, a reduced oxidant or YO is reoxidized into an oxidant or an oxygen insertion agent. Preferred reoxidizing agents are air or dioxygen (O₂). Suitable oxidants are those that can be reoxidized with air in a thermodynamically favorable reaction: Y+½O₂→YO where ΔGᵣₓₙ <0 kcal/mol at temperatures below 300° C. On the basis of tabulated data, the following examples are given by way of example only.

SeO₄²⁻+CH₄→SeO₃²⁻+CH₃OH AG=−12 kcal/mol at 250° C., K=10⁵

SeO₃²⁻+½O₂→SeO₄²⁻ ΔG=−14 kcal/mol at 250° C., K=10⁵

NO₃⁻+CH₄→NO₂⁻+CH₃OH ΔG=−11 kcal/mol at 250° C., K=10⁴

NO₂⁻+½O₂→NO₃⁻ΔG=−15 kcal/mol at 250° C., K=10⁶

CH₃S(O)CH₃+CH₄→CH₃OH+CH₃SCH₃ ΔG=−2 kcal/mol at 250° C., K=6

CH₃SCH₃+½O₂→CH₃S(O)CH₃ ΔG=−17 kcal/mol at 250° C., K=10⁷

The term “releasing an oxidized hydrocarbon” refers to a step during a process for selectively oxidizing hydrocarbons as disclosed herein. During this step, an oxidized hydrocarbon is released from a metal.

The term “selectively oxidizing” refers to C—H bond selectivity exhibited by a catalyst during C—H bond activation and subsequent steps. Selective oxidation occurs for example when a catalyst selects a primary versus a secondary or tertiary C—H bond. Selectivity can also occur when a catalyst selects an alkyl C—H bond of an unreacted hydrocarbon versus that of an oxidized or functionalized hydrocarbon.

The term “solid support” refers to an insoluble matrix to which a catalyst or catalyst complex is attached. An example is an ion exchange resin. Other examples include but are not limited to metal oxides such as magnesium oxide, calcium oxide, and barium oxide as well as potassium fluoride on alumina and some zeolites.

The term “solvent assisted” refers to the role a solvent molecule plays in reaction energetics of a C—H bond activating step. As an example, a consequence of solvent assistance can be an increased reaction rate for the C—H bond activating step and an overall increase in the hydrocarbon oxidation process.

Suitable oxidant species include but are not limited to oxygen (O₂), and ozone (O₃). Suitable oxidants also include amine oxides (R₂N—O), such as, but not limited to pyridine-N-oxide, morpholine-N-oxide, trimethyl amine-N-oxide, triethyl amine-N-oxide, nitrile oxides, mesityl nitrile oxide ((CH₃)₃C₆H₃-N—O), acetylnitrile oxide.

Other suitable oxidants are peroxides and peracids (ROOR and ROOCOR), including but not limited to hydrogen peroxide (HOOH), alkyl peroxides (ROOR), disilylperoxides (R₃SiOOSiR₃).

Other suitable oxidants include chlorinated oxo species, including but not limited to dichlorine monoxide (ClOCl), sodium hypochlorite (NaOCl), hypochlorite (HOCl). Other suitable oxidants are iodosylarenes (RₓC₆H₆-x-IOₙRₙ), including but not limited to iodosylbenzene (C₆H₆-IO₂), iodosylacetate (C₆H₆-I(OAc)₂).

Other suitable oxidants are various oxides of selenium, sulfur, phosphorus, and tellurium.

Suitable transalkylation agents include those reagents capable of accepting alkyl groups and converting the transferred alkyl groups to alcohols, amines, or similarly functionalized products. Suitable transalkylation agents receive an alkyl group from a suitable donor M-R species and have a functionalization pathway that are unavailable or more facile than for the M-R bond itself, typically, but not exclusively, a pathway involving reductive functionalization or oxygen atom insertion/hydrolysis. Suitable transalkylation agents include, but are not limited to HgX₂, SeOX₂, SeO₃²⁻, CuX, CuX₂, CuX₂, TeX₄, TeOX2₂, SbX₃, SnX₄, SnO₂, AgX, PbX₄, ZnX₂, AuX, where X=OH, alkyoxy, halide, acetate, halogenated acetate, or the conjugate base of the solvent, and derivatives thereof.

An advantage of systems which activate and functionalize hydrocarbons in weakly acid, neutral and basic media is that such systems should not be as severely inhibited by Lewis basic products such as H₂O or CH₃OH.

With the move to less electrophilic electron-rich metal complexes, the pathway for M-R functionalization that operates for the electrophilic Pt(II) and Hg(II) systems may not be available in acidic or weakly basic media. The nature of the metal species and surrounding ligands renders the metal relatively electron rich (nucleophilic) compared with electrophilic catalysts operating in acidic media. Catalysts which activate and functionalize hydrocarbons in weakly acidic, neutral and basic media produce M-R species having electron-rich (nucleophilic) metal carbon bonds where the carbon group is not very electrophilic. Thus, while these species are not likely to under facile reductive functionalization by attach of nucleophiles on the carbon group, it is possible that such species will be susceptible to attack of electrophilic species. This can be understood in the comparison of the relatively facile reactions of CF₃CO₂⁻ on the methyl group of [CH₃O(CH₃)₂]+ which generates CH₃OC(O)CF₃ while attack of CF₃CO₂⁻ on the methyl group of CH₃-B(CH₃)₂ to generate CH₃OC(O)CF₃ and (CH₃)₂B⁻ is not observed. This results from a combination of factors: A) because the methyl groups on CH₃-B(CH₃)₂ are negatively instead of positively polarized and B) because the reaction with CH₃-B(CH₃)₂ (as a result of the low electronegativity of B) would be thermodynamically unfavorable. To convert the Me-B bond of CH₃-B(CH₃)₂ to a Me-O bond requires reaction with an oxygen species capable of becoming formally positively charge and where the reaction is thermodynamically favorable. Such a reaction is possible with O-atom species attached to relatively electron-withdrawing species such as HO—O⁻, Py-O, IO₄⁻, IO₃⁻, SeO₄²⁻, etc. In all of these YO cases, CH₃-B(CH₃)₂ can be made to react to generate CH₃-O-B(CH₃)₂ species by donation of the O-atom from the YO. In these cases, the reactions are facilitated by the generation of formal positive charge on the donated O-atom in the transition state for reaction and the favorable thermodynamics resulting from loss of relatively good leaving groups and a stable CH₃-O-B(CH₃)₂ where the boron remains bound to three species. In a similar manner, different M-CH₃ can take on the character of CH₃Cl or CH₃-B(CH₃)₂ depending on the electron-accepting and electron-donating characteristics, respectively, of the M species.

Without being bound to theory, the reactivity of a M-R species in weakly acidic, neutral and basic media to yield a M-OR species can be viewed as a formal electrophilic attack on the carbon of the M-R group. Such “electrophilic cleavage” has long been known as a way to cleave electron-rich metal carbon bonds. Electrophilic cleavage occurs in part because the M-C bond polarization may be depicted as M^{δ+}-^{δ-}-R. Such polarization increases the negative charge on carbon.

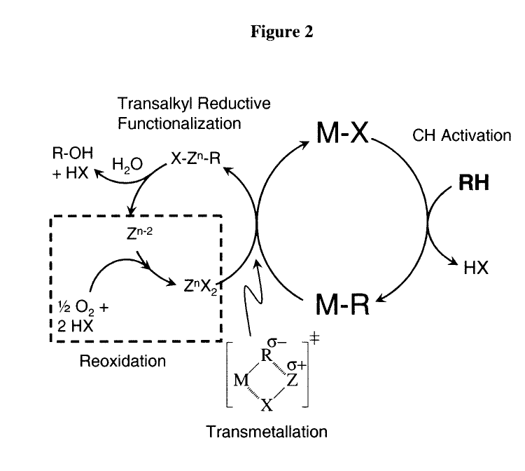

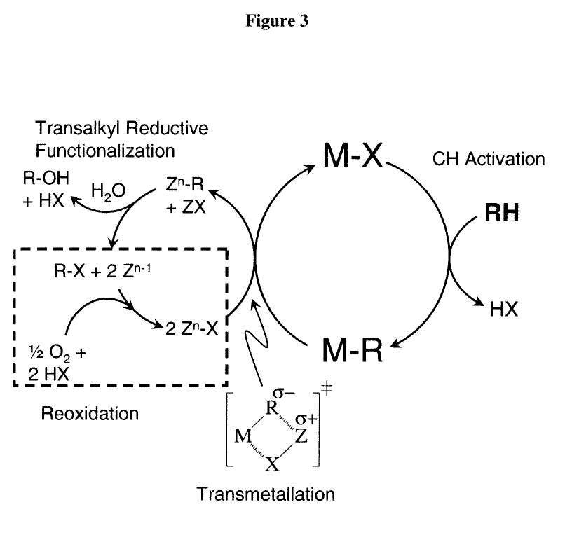

In contrast to M-R species generated in highly acid media, M^{δ+}-^{δ-}R polarized intermediates generated from CH activation reactions with electron rich M-R species are unlikely to undergo functionalization by reductive processes involving attack of nucleophilic oxygens even with species as nuclophilic as OH⁻ on the polarized M-R^{δ-} intermediates. Because reactions that generate M-R intermediates are likely to be carried out in the presence of OH⁻, it is desirable to identify pathways and reagents to facilitate functionalization reactions based on attack by OH⁻. As shown in FIGS. 2 and 3, to maximize M-R^{α-} polarization and efficient functionalization, catalytic systems which activate and functionalize hydrocarbons in weakly acid, neutral and basic media catalysts should be based on electropositive or electron-donating metals (e.g. Os(II)) in electropositive (or Lewis basic) solvents such as OH⁻ in H₂O. In addition, reactivity considerations of polar bonds predict that efficient functionalization of M^{δ+}-^{δ-}R polarized intermediates can be efficiently accomplished by electrophilic attack with positively polarized oxygen atoms, Y^{δ-}-^{δ+}O (not by nucleophilic attack with negatively polarized oxygen atoms).

Additionally, because the M-R intermediate, and consequently the CH cleavage transition state leading to this intermediate, are also M^{δ+}-^{δ-}R polarized, then electron donating groups (EDG) on the carbon group undergoing the CH cleavage can destabilize the CH activation transition state. This is desirable, since this with such catalysts methanol in basic media can be protected and potentially made less reactive than methane. Methanol in basic media will hydrogen bond as MeOH-OH⁻ (this interaction can be viewed as resonance contributions):

MeOH-OH⁻←→MeO⁻ HOH (Eq. 1)

The hydrogen bonding interaction to OH- increases the electron density at carbon, i.e., renders a CH bond in methanol product less susceptible to reaction compared to reactions of the CH bond in an alkane if the catalyst cleaves the CH bonds in transition states that lead to build up of negative charge on the carbon group. This concept is important as this could allow the methanol product to be protected from subsequent over-oxidation. As an example of this concept it should be obvious to those skilled in the art that the reaction of methane and methoxide with a strongly basic species such as the oxide (O²⁻) anion should lead to more deprotonation of methane than the CH bonds on CH₃O⁻. This is because the O⁻ group attached to the methyl group in CH₃O⁻ is more electron-donating than H attached to the methyl group in methane. Additionally, since deprotonation of a CH bond of methane by a strong base, such as O²⁻, is an extreme example of nucleophilic CH activation to generate OH⁻ and a fully charged species CH₃⁻, the presence of the electron-donating group O⁻ in CH₃⁻ will minimize deprotonation (nucleophilic CH activation) of the CH group by O²⁻ to generate OH⁻ and [CH₂O]²⁻. In the base of transition metal hydroxide that react with CH bonds by nuclephilic substitution reactions to generate M-CH₃ and H₂O via transition states that generate negative charge on the carbon, similar protecting effects that destabilize the transition state for CH activation of CH₃O⁻ relative to CH₄ would be expected.

Solvent also be expected to play a role in the functionalization of M-R intermediates generated with catalysts which activate and functionalize hydrocarbons in weakly acid, neutral and basic media catalysts. In the present case, the solvent induces M^{δ+}-^{δ-}CH₃ polarization as well as increase the thermodynamic driving force for reaction with electrophilic oxidants polarized as Y^{δ-}-^{δ+}O. Such M^{δ+}-^{δ-} CH₃ polarization is achieved in neutral and basic media catalysts by HO⁻ interaction between the ligands coordinated to the electropositive, low oxidation state metal complexes.

In addition to electrophilic oxidants (O-atom donors) it is possible to carry out functionalization with other electrophiles. One class of such electrophiles are species that are capable of redox reactions (species that can thermodynamically favorably undergo changes in formal oxidation states under the reaction conditions).

The term “transalkylation to” refers to a chemical process wherein an alkyl group is transferred from an alkane activating metal species to an alkyl acceptor which serves as an alternative redox center and which then undergoes reductive functionalization. Such a pathway transfers the alkyl group from the M-R intermediate to another species (X-Z-R) as shown in FIG. 2. The intermediate species X-Z-R is then capable of undergoing reduction functionalization. This is shown in FIGS. 2.

In view of theoretical support, H₂SeO₃ plays the role of an electrophilic Z-X alkyl receptor that is attacked by the carbon-based HOMO of the polarized M-R intermediate. Alkyl transfer occurs, likely via the transalkylation transition states shown in FIGS. 2 and 3, to generate Z-R intermediates that are subsequently oxy-functionalized via a reductive process to generate R-OH.

In view of theoretical support, suitable alkyl acceptors species include but are not limited to Cu(II), Se(IV), Se(VI), Ni(II), Fe(III), Mn(III), V(IV), Hg(II), Ag(I), Pd(II), Au(I), Au(III), etc. In view of theoretical support, any species that is less electron-withdrawing than M in the M-R intermediate or more specifically, where the reaction shown in Eq 2, proceeds with an overall barrier (from the resting state of the oxidation system) of no greater than 35 kcal/mol. Suitable examples of alkyl acceptors are species where Equation 2 is both thermodynamically and kinetically favorable (with barriers <15 kcal/mol).

M-R+Z-X→M-X+Z-R (2)

Z-R subsequently undergoes facile functionalization to R-X (for example hydrolysis to X=OH). One efficient method of such functionalization reaction is nucleophilic attack of an O-nucleophile on electrophilic carbon, thus solvent (⁻OH) attack is a particularly likely method for functionalization of Z-R.

Another aspect of Z-X and its role in assisting functionalization is the feasibility (thermodynamically and kinetically) of reoxidizing the reduced species, Zⁿ⁻² or Zⁿ⁻¹ (in FIGS. 2 and 3) back to the oxidized Z-X or ZX₂ species.

Yet another aspect of Z-X is that it should be stable to solvent and product methanol under reaction conditions. Thus Z-X should not rapidly react with methanol or at least should only react with methanol in the reaction system at a rate lower than the methanol is produced.

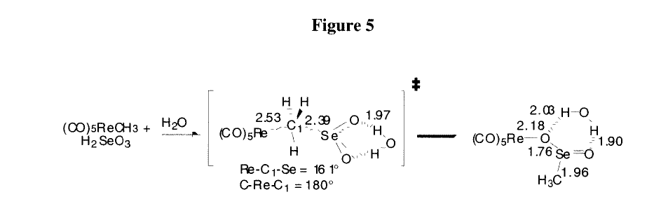

The treatment of CH₃Re(I)(CO)₅ in acetonitrile-water mixtures with SeO(OH)₂ readily leads to the formation of CH₃SeO(OH) in almost quantitative yields. This reaction most likely proceeds via the transalkylation transition state shown in FIG. 5.

A series of experiments showed that the reaction of CH₃Re(CO)₅ with O-atom transfer agents to produce methanol was feasible. The direct reaction of CH₃Re(CO)₅ with three different terminal oxidants was explored, namely, PhIO, PyO and IO₄⁻ in 9:1 water/acetonitrile solution. Both PhIO and IO₄⁻ were efficient for generation of methanol, however not particularly selective (30 ±6% yield of methanol with PhIO, and 20 ±2% using KIO₄). Control experiments showed that the reaction rates and selectivities are independent of added O₂ and free-radicals were likely not involved.

The functionalization of the Re(I)-CH₃ with Se(IV) is clean and facile. Carrying out the reaction of CH₃Re(CO)₅ with D₂SeO₃ (generated in situ from SeO₂ and D₂O) in a solution of CD₃CN and D₂O produced CH₃SeO₂D in quantitative yield as identified by comparison of the ¹H and ¹³C NMR spectra and mass peaks to that of the commercially available authentic sample. No carbon dioxide, which is often produced in reactions of CH₃Re(CO)₅ with oxidants, and indicates overoxidation, was identified in gas chromatography-mass spectrometry (GC-MS) analyses of the headspace of the reaction mixture.

This seleno-functionalization reaction proceeds cleanly despite the possibility of side reactions. Additionally, the observation that the reaction of this Se(IV) proceeds in high yield shows this reaction is substantially different from the corresponding direct reactions with O-atom transfer agents. Without being bound by theory the increased polarizability of the Se(IV) center relative to the oxygen center of the other YO reactants could partially account for the high selectivity observed for this functionalization reaction.

As shown in FIG. 5, transalkylation can be used catalytically with an added oxidant. In that case, the net reaction is the conversion of methane to methanol.

The addition of an oxidant capable of converting the CH₃SeO₂D to methanol and H₂SeO₃ allowed the reaction to proceed with catalytic amounts of SeO₂. The catalytic functionalization of this complex via a catalytic amount of Se(IV) (0.1 equivalent) and an oxidant (excess KIO₄) in aqueous media at 100° C. was examined. The CH₃Re(CO)₅ system was stable over the time period studied (12 h) and although production of methane in the absence of Se(IV) or the oxidant was noted, especially at higher temperatures or longer reaction times, no methane was observed in the catalytic system. In the reaction system, CH₃SeO₂D was observed to be formed before the production of methanol. Although periodate was found to convert CH₃Re(CO)₅ to methanol without the use of Se(IV), when a catalytic amount of H₂SeO₃ was used the selectivity of the overall reaction increases from approximately 20 to an 80% yield in methanol.

In addition, CD₃OSeO₂D (produced from CD₃OD and SeO₂) was also found to produce CH₃Se(O)CD₃ in high yield (79.3%).

Conversion of Sn(CH₃)₄ to CH₃SeO₂H and HOSn(CH₃)₃ with H₂SeO₃ in acetonitrile/water at 100° C. for 30 minutes was also found to be facile. Reaction of CH₃Re(CO)₅ with H₂SeO₃ as Oxidant.

All reactions were carried out in 9:1 CD₃CN/D₂O in 8″ NMR tubes equipped with resealable J-Young Teflon valves. Tetramethyl tin (140 mg, 0.1 mmol) was charged into an NMR tube, followed by approximately 1 equivalent of SeO₂ (9.76 mg, 0.088 mmol), followed by CD₃CN and D₂O (9:1, 0.7 mL added), along with 0.6 μL of cyclohexane for use as an internal standard. All appropriate blanks were taken to assign solvent peaks, starting material peaks, and product (CH₃SeO₂H and HOSn(CH₃)₃ formation. Reactions were typically carried out under air at 100° C. for 30 minutes. Yield of CH₃SeO₂ appeared quantitative by ¹H NMR. CH₃SeO₂H(D) ¹H NMR (9:1 CD₃CN/D₂O): δ2.60(s, 3H, Se-CH₃, ²J_{Se-H}13.2 Hz), and HOSn(CH₃)₃ δ0.2.

The results presented herein represent a mild and highly selective pathway for functionalization of electron-donating metal alkyl (M-R) intermediates via a pathway with catalytic transalkylation agents and an oxidant.

Se(IV) catalyzed functionalization of M-R intermediates represents one example of air-recyclable YOs and M-Rs and can be extended to other ligand sets and electronic configurations.

Integrating the reactions disclosed with the CH activation reaction allows a catalytic cycle to be developed for the selective conversion of methane to methanol with catalysts that are not inhibited by methanol or water.

In conclusion, herein is reported evidence for the facile conversion of Re-CH₃ to methanol by reaction with Se(IV), and an O-atom donor.

The extension of use of other transalkyation reagents based on S, Te, Sn, or B.

These are the first known examples of functionalization of electron-rich M-R species to generate R-heteroatom products and alcohols by non-radical reaction mechanisms.

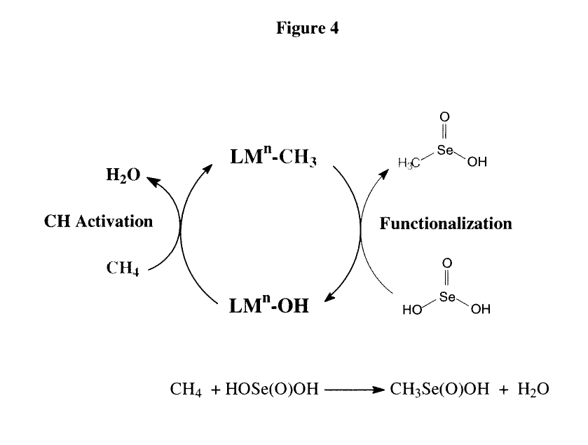

Coupling a transalkylation reaction involving selenium (IV) and a catalyst that activated methane in weakly acid, neutral and basic media, conceptually leads to a process for the conversion of methane to a methyl selenium species as shown in FIG. 4. In view of theoretical support, the net reaction is thermodynamically favorable and is a potential functionalization reaction.

### EXAMPLES

General Considerations: All air and water sensitive procedures were carried out either in a Vacuum Atmospheres inert atmosphere glove box under argon, or using standard Schlenk techniques under argon. Methyl iodide (Aldrich) was used as purchased. The labeled methyl iodide, ¹³CH₃I, (Cambridge Isotopes) was used as purchased. Rhenium carbonyl (Re₂CO₁₀) was purchased from Strem. Selenium(IV) oxide (Research Organic/Inorganic Chemical Corp.) was used as purchased. GC/MS analysis was performed on a Shimadzu GC-MS QP5000 (ver. 2) equipped with cross-linked methyl silicone gum capillary column (DB5). The retention times of the products were confirmed by comparison to authentic samples. All NMR spectra were obtained on a Varian Mercury-400 spectrometer at room temperature. All chemical shifts are reported in units of ppm and referenced to the residual protic solvent.

### Example 1

### Reaction of CH₃Re(CO)₅ with PhIO as oxidant

(CO)₅ReCH₃+PhIO: All reactions were carried out in 9:1 CD₃CN/D₂O in 8″ NMR tubes equipped with a resealable J-Young Teflon valve. Approximately 30 mg (0.088 mmol) of methyl rhenium(I) pentacarbonyl was charged to the NMR tube, followed by 3 equivalents of PhIO (58.08 mg, 0.264 mmol), followed by acetone-d₆ (0.7 mL added), along with 0.6 μL of cyclohexane for use as an internal standard. All appropriate blanks were taken to assign solvent peaks, starting material peaks, and product (methanol) formation. Reactions were typically carried out under air at 100° C. for 4 h. ¹H NMR indicated a 30 ±6% yield of methanol.

### Example 2

### Reaction of CH3Re(CO)5 with KIO4 as oxidant

All reactions were carried out in 9:1 CD₃CN/D₂O in 8″ NMR tubes equipped with a resealable J-Young Teflon valve. Approximately 10 mg (0.088 mmol) of methyl rhenium(I) pentacarbonyl was charged to the NMR tube, followed by 3 equivalents of KIO₄ (60.24 mg, 0.262 mmol), followed by CD₃CN and D₂O (9:1, 0.7 mL added), along with 0.6 μL of cyclohexane for use as an internal standard. All appropriate blanks were taken to assign solvent peaks, starting material peaks, and product (methanol) formation. Reactions were typically carried out under air at 100° C. for 12 h. ¹H NMR indicated a 20 ±2% yield of methanol.

### Example 3

### Reaction of CH3Re(CO)5 with H2SeO3 as oxidant

All reactions were carried out in 9:1 CD₃CN/D₂O in 8″ NMR tubes equipped with a resealable J-Young Teflon valve. Approximately 30 mg (0.088 mmol) methyl rhenium(I) pentacarbonyl was charged to the NMR tube, followed by 1 equivalent of SeO₂ (9.76 mg, 0.088 mmol), followed by CD₃CN and D₂O (9:1, 0.7 mL added), along with 0.6 μL of cyclohexane for use as an internal standard. All appropriate blanks were taken to assign solvent peaks, starting material peaks, and product (methanol) formation. Reactions were typically carried out under air at 100° C. for 30 minutes. Yield of CH₃SeO₂H appeared quantitative by ¹H NMR. CH₃SeO₂H(D) ¹H NMR (9:1 CD₃CN/D₂O): δ 2.65(s, 3H, Se-CH₃, ²J_{Se-H} 13.2 Hz). ¹³C{¹H} NMR (9:1 CD₃CN/D₂O): δ 42.2(Se-CH₃, ¹J_{Se-H} 90.2 Hz).

### Example 4

### Reaction of CH3Re(CO)5 with catalytic amount of H2SeO3 and KIO4 oxidant

(CO)₅ReCH₃+0.1 H₂SeO₃+3 KIO₄: All reactions were carried out in 9:1 CD₃CN/D₂O in 8″ NMR tubes equipped with a resealable J-Young Teflon valve. Approximately 30 mg (0.088 m/mol) methyl rhenium(I) pentacarbonyl was charged to the NMR tube, followed by approximately 0.1 equivalent of SeO₂ (1.0 mg, 0.009 mmol), KIO₄ (60.72 mg, 0.264 mmol), and finally CD₃CN and D₂O (9:1, 0.7 mL added), along with 0.6 μL of cyclohexane for use as an internal standard were added. All appropriate blanks were taken to assign solvent peaks, starting material peaks, and product (methanol) formation. Reactions were typically carried out under air at 100° C. for 30 minutes.

1. A process for the production of an alcohol comprising:
(a) contacting a metal complex comprising an alkyl moiety with an alkyl acceptor, thereby alkylating the alkyl acceptor;
(b) contacting the alkylated acceptor with an oxidant, thereby producing an alcohol and regenerating the alkyl acceptor.

2. The process of claim 1, wherein the metal complex comprises a group 7, group 8, or group 9 transition metal, selected from the group consisting of rhenium, ruthenium, osmium, rhodium, and iridium.

3. The process of claim 2, wherein the metal complex comprises rhenium (I).

4. The process of claim 1, wherein the alkyl acceptor comprises an element chosen from the group consisting of selenium, copper, iron, nickel, manganese, vanadium, mercury, platinum, palladium, and silver.

5. The process of claim 1, wherein the alkyl acceptor is a selenium oxo species.

6. The process of claim 5, wherein the selenium oxo species is selenic acid, selenous acid, or selenium dioxide.

7. The process of claim 1, wherein the molar amount of the alkyl acceptor is substantially less than the molar amount of the oxidant.

8. The process of claim 7, wherein the oxidant is an O-atom donor.

9. The process of claim 8, wherein the O-atom donor is selected from the group consisting of iodate, periodate, or mixtures of iodine and oxygen.

10. A process for the selective oxidation of an alkane, the process comprising:
(a) contacting an alkane with a CH activating metal complex and an alkyl acceptor, thereby producing a metal complex comprising an activated alkyl;
(b) transfering the activated alkyl to the alkyl acceptor, thereby alkylating the alkyl acceptor, and regenerating the CH activating metal complex;
(c) producing an alcohol by contacting the alkylated acceptor with an oxidant and regenerating the alkyl acceptor.

11. The process of claim 10, wherein the CH activating metal complex comprises a group 7, group 8, or group 9 transition metal.

12. The process of claim 11, wherein CH activating metal complex comprises a metal selected from the group consisting of rhenium, ruthenium, osmium, rhodium, and iridium.

13. The process of claim 10, wherein the alkyl acceptor comprises an element chosen from the group consisting of selenium, copper, iron, nickel, manganese, vanadium, mercury, platinum, palladium, and silver.

14. The process of claim 10, wherein the molar amount of the alkyl acceptor is substantially less than the molar amount of the oxidant.

15. The process of claim 10, wherein the alkyl acceptor comprises selenium.

16. The process of claim 15, wherein the alkyl acceptor is a selenium (IV) oxo species.

17. The process of claim 16, wherein the selenium (IV) oxo species is selenic acid or selenium dioxide.

18. The process of claim 1, wherein the oxidant is periodate.
